(19) United States
(12) Patent Application Publication
(10) Pub. No.: US 2010/0210994 A1
(43) Pub. Date: Aug. 19, 2010

(54) DELIVERY OF THERAPEUTICALLY/DERMATOLOGICALLY ACTIVE SPECIES INTO THE SKIN VIA ELECTROPORATION

(75) Inventor: Leila Zarif, Peymeinade (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/541,376

(22) Filed: Aug. 14, 2009

Related U.S. Application Data
(63) Continuation of application No. PCT/EP2008/052081, filed on Feb. 20, 2008.

(51) Int. Cl. A61N 1/30

(52) U.S. Cl. 604/20

(57) ABSTRACT

A regime or regimen for treating a skin disease, disorder or condition afflicting a patient in need thereof includes forming at least one micropore to a predetermined depth through a surface of the afflicted skin of such patient; positioning at least a first electrode on the surface of the afflicted skin electrically coupled to the at least one micropore and a second electrode on the surface of the afflicted skin spaced apart from the first electrode; applying an electrical voltage from the first and second electrodes to produce a desired electroporation in the skin, and further including the step of delivering a biologically active species to the afflicted skin at the at least two micropores formed therein, wherein the biologically active species is therapeutically/dermatologically active against the skin disease, disorder or condition.

### CROSS-REFERENCE TO PROVISIONAL/PCT APPLICATIONS

This application claims priority under 35 U.S.C. §120 of U.S. Provisional Application No. 60/902,088, filed Feb. 20, 2007, and is a continuation/national phase of PCT/EP 2008/052081, filed Feb. 20, 2008 and designating the United States (published in the English language on Aug. 28, 2008 as WO 2008/101968 A2) each hereby expressly incorporated by reference in its entirety and each assigned to the assignee hereof.

### BACKGROUND OF THE INVENTION

1. Technical Field of the Invention

The present invention relates to a regime or regimen for treating a skin disease, by delivery of therapeutic biologically active substances into the skin.

2. Description of Background and/or Related and/or Prior Art

Electroporation of tissue, such as skin, is employed to enhance the permeability of the tissue, or delivery of substances to, the tissue. Electroporation used alone for enhancing the permeability of tissue, such as skin, has limited applications and utility.

Other techniques for enhancing the permeability of tissue surfaces have been developed. One such technology is the microporation of tissue, wherein the tissue surface, such as the skin or mucosal layer, is physically breached by the formation of micropores.

U.S. Pat. No. 6,022,316 is directed to an apparatus and method for electroporating tissue.

At least one micropore is formed to a predetermined depth through a surface of the tissue; first and second electrodes are positioned spaced apart on the tissue and one of the electrodes is electrically coupled to the at least one micropore; and an electrical voltage is applied from the electrodes to produce a desired electroporation in the tissue from the electrodes.

### SUMMARY OF THE INVENTION

Briefly, the present invention features utilization of apparatus as described in U.S. Pat. No. 6,022,316 for treating skin diseases such as acne or psoriasis.

Thus, it has now been discovered that such apparatus is effective for enhancing penetration of the substances in or though the skin, specifically for those which penetrate only slightly into skin.

At least one micropore is formed to a predetermined depth through a surface of the tissue; first and second electrodes are positioned spaced apart on the tissue and one of the electrodes is electrically coupled to the at least one micropore; and an electrical voltage is applied from the electrodes to produce a desired electroporation in the tissue from the electrodes. The electroporation electrodes may also serve the function of participating in the microporation of the tissue. In accordance with a preferred embodiment, a device having elements that are suitable for microporating the skin and electroporating the skin is provided.

By microporating the tissue prior to the application of electroporation, the parameters for electroporation can be significantly adjusted and the sensation to the patient can also be reduced. Furthermore, by first breaching the surface of the tissue with micropores, the electroporation can be directed at selected structures in the skin tissue matrix, such as capillaries. In addition, electroporation applied to the capillaries also increases the capillary permeability to substances which are to be delivered into the tissue.

This invention features a method for electroporating tissue, comprising the steps of:

a) forming at least one micropore to a predetermined depth through a surface of the tissue;

b) positioning at least a first electrode on the surface of the tissue electrically coupled to the at least one micropore and a second electrode on the surface of the tissue spaced apart from the first electrode; and

c) applying an electrical voltage from the first and second electrodes to produce a desired electroporation in the tissue.

The method of the invention is useful for treating a skin disease and further comprises the step d) of delivering a substance to the skin at the at least two micropores formed therein, wherein the substance is therapeutically active against a skin disease.

Preferably, the step of applying electrical voltage comprises applying an electrical voltage of a sufficient magnitude from the first and second electrodes suitable to produce a potential drop exceeding a nominal threshold to achieve electroporation across the epithelial cell layer but not sufficient to electroporate membranes present in other tissue structures thereby achieving a selective electroporation of targeted membranes.

Step a) may comprise forming first and second micropores spaced apart from each other, and wherein the first electrode is positioned to be electrically coupled to the first micropore, and the second electrode is positioned to be electrically coupled to the second micropore.

Step c) may comprise applying a voltage pulse of a first polarity with respect to the first and second electrodes, followed by a voltage pulse of an opposite polarity with respect to the first and second electrodes.

Step a) may comprise forming a plurality of micropores spaced apart from each other in the tissue, and step b) comprises placing a plurality of electrodes each being electrically coupled to a different one of the micropores, and wherein step c) comprises applying electrical voltage pulses from different sets of the plurality of electrodes so as to electroporate the tissue in multiple directions. In this particular embodiment, step c) may comprise applying a voltage pulse of a first polarity from a first set of electrodes followed by a voltage pulse of an opposite polarity from the first set of electrodes.

The apparatus which is useful for microporation and electroporation of skin comprises:

(a) a heated probe suitable for conducting heat to a surface of the skin to form at least one micropore therein;

(b) at least first and second electrodes suitable for being spaced apart from each other on the skin; and

(c) control means for supplying energy to the heated probe so as to cause formation of the at least one micropore in the skin, and for applying an electrical voltage from the first and second electrodes suitable for electroporating the skin.

Preferably, the control means supplies a magnitude of electrical voltage applied from the first and second electrodes suitable to produce a potential drop exceeding a nominal threshold to achieve electroporation across the epithelial cell layer but not sufficient to electroporate membranes present in other skin structures thereby achieving a selective electroporation of targeted membranes.

In a particular embodiment, the heated probe forms first and second micropores spaced apart from each other in the tissue, and wherein the first electrode is suitable for being electrically coupled to the first micropore and the second electrode is suitable for being electrically coupled to the second micropore.

Preferably, the heated probe is an electrically heated probe, and wherein the control means supplies electrical current to the electrically heated probe to form the at least one micropore.

The electrically heated probe may also serve as the first electrode such that the electrical voltage is applied from the electrically heated probe and the second electrode.

In another particular embodiment, the heated probe comprises first and second electrically heated probes spaced apart from each other each responsive to electrical current supplied by the control means to form two micropores in the tissue spaced apart from each other.

Preferably, the first and second electrically heated probes further serve as the first and second electrodes, the control means being coupled to the first and second electrically heated probes so as to apply the electrical voltage therefrom.

Such apparatus may further comprise a skin-contacting layer supporting the first and second electrically heated probes, and further comprising conductor means for coupling electrical current from the control means to the first and second electrically heated probes, and for applying the electrical voltage from the first and second electrically heated probes.

In a particular embodiment, the control means applies a first voltage pulse of a first polarity with respect to the first and second electrodes, followed by a voltage pulse of an opposite polarity with respect to the first and second electrodes.

The apparatus may comprise a plurality of electrically heated probes each responsive to electrical current and suitable for forming a plurality of micropores spaced apart from each other in the tissue, and wherein the control means applies electrical voltage pulses from different sets of the plurality of electrically heated probes so as to electroporate the tissue in multiple directions. In this embodiment, the control means preferably applies a voltage pulse of a first polarity from a first set of electrodes followed by a voltage pulse of an opposite polarity from the first set of electrodes.

In another embodiment, the apparatus comprises a mechanical element suitable for causing the surface of the skin to sufficiently bulge from the first and second electrodes to place tissue structures desired to be electroporated in a principal current path from the first and second electrodes.

The apparatus may further comprise means for applying suction to the tissue so as to suck the surface of the tissue from the first and second electrodes.

The above and other objects and advantages of the present invention will become more readily apparent when reference is to made to the following description, taken in conjunction with the accompanying drawings.

### BRIEF DESCRIPTION OF THE DRAWINGS

FIG. 1 is a flow chart generally depicting the overall process employing microporation and electroporation of tissue in accordance with the present invention.

FIG. 2A is a schematic diagram of an apparatus for electroporating tissue according to the present invention.

FIG. 2B is a schematic diagram showing the coupling of electrical current for microporation and electrical voltage for electroporation are supplied to combination electrical heated probes/electroporation electrodes.

FIG. 3 is an enlarged longitudinal cross-sectional view of a device suitable for use in microporating and electroporating tissue.

FIG. 4 is a bottom view of the device of FIG. 3, showing the electrically heated probes used for microporating and electroporating tissue.

### DETAILED DESCRIPTION OF BEST MODE AND SPECIFIC/PREFERRED EMBODIMENTS OF THE INVENTION

### Definitions

As used herein, “poration,” “microporation,” or any such similar term means the formation of a small hole or pore to a desired depth in or through the skin. Preferably the hole or micropore will be no larger than about 1 mm (1000 μm) in diameter, and will extend to a selected depth, as described hereinafter.

“Electroporation” means a process by which electrical current is applied through skin by electrodes spaced apart on or in the skin to temporarily increase the permeability of the skin to collection of fluids therefrom, or delivery of permeants thereto. It involves the delivery of pulses of electrical energy of relative short duration to cause the voltage potential developed across the targeted skin structure to be sufficiently greater than a threshold level to produce the desired electroporation. The parameters typical of electroporation and the thresholds for effective operation under many operating conditions are well known in the art, and are discussed in several articles, including “Electroporation Of Mammalian Skin: A Mechanism to Enhance Transdermal Drug Delivery,” Proc. Nat'l Acad. Sci., 90:1054-1058 (1993) by Prausnitz et al., and “Methods For In Vivo Tissue Electroporation Using Surface Electrodes,” Drug Delivery, 1:1265-131 (1993) by Prausnitz et al.

As used herein, “micropore” or “pore” means an opening formed by the microporation method.

The term “heated probe” means a probe, preferably solid phase, which is capable of being heated in response to the application of electrical or electromagnetic (optical) energy thereto. For simplicity, the probe is referred to as a “heated probe” which includes a probe in a heated or unheated state, but which is heatable.

The present invention is directed to creating an electroporation effect to selectively enhance the permeability of selected structures within skin, including but not limited to, cell membrane walls, the membranes separating different tissue types and the walls of the capillaries and blood vessels present in the dermis, to allow a greater out-flux of the aqueous fluid from within the blood volume into the interstitial spaces or to allow a greater influx of a compound introduced into these surrounding tissues and hence the blood stream.

It is known that the physical size of the capillary and vessel cross-section is several times larger than the dermal and epidermal cells also present in the current path, and the potential drop across all of these structures is known to occur almost exclusively at the outer membrane, or in the case of the capillaries or blood vessels, at the epithelial cell layer comprising the main barrier structure within the wall of the capillary or vessel. Consequently, a current density sufficient to produce a potential drop exceeding the nominal threshold (preferably greater than about 1 volt) to achieve electroporation across the epithelial cell layer is not sufficient to electroporate the membranes present in other tissue structures, such as the cell walls of the epidermal cells, through which the current is flowing, hence allowing a selective electroporation of only the targeted membranes.

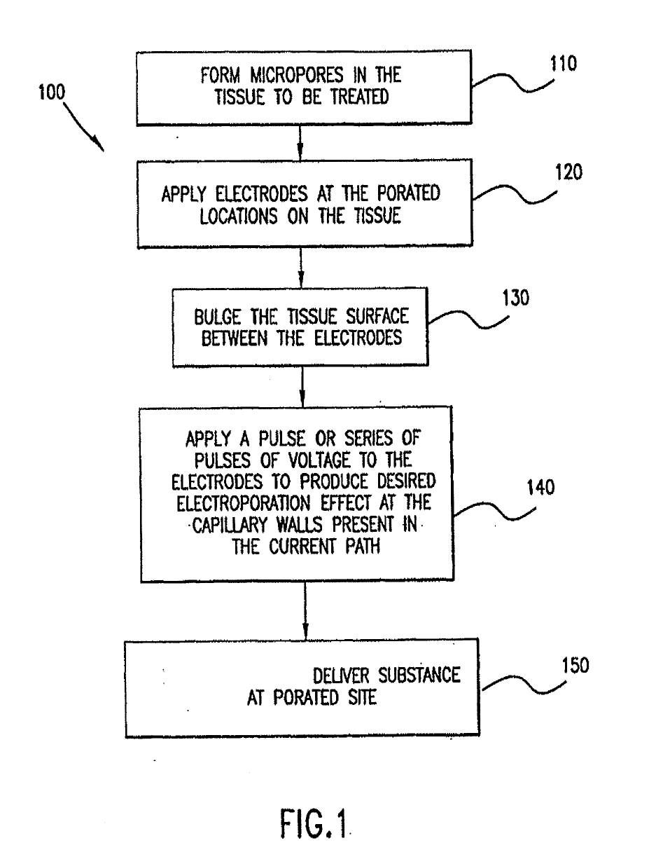

FIG. 1 depicts the steps of the overall process 100 according to the present invention. Various devices and techniques for performing each of the steps in FIG. 1 are shown and described hereinafter. Briefly, the overall process involves forming at least one micropore to a predetermined depth range through a surface of the skin; positioning at least a first electrode electrically coupled to the at least one micropore and a second electrode spaced apart from the first electrode; applying an electrical voltage from the first and second electrodes sufficient to produce a desired electroporation in the skin present in the induced current path.

Step 110 involves forming micropores in the skin to be treated. At least one micropore is formed, though as will become more apparent hereinafter, multiple micropores may be formed. The micropore is formed through a surface of the skin to a predetermined depth range into the skin. For example, at least one microporation in the outer layer of the epidermis is formed to allow the high impedance layer of the stratum corneum to be eliminated from the current path.

Preferably, at least two micropores are formed some distance apart in the locations where the electrodes are to be placed. The micropores range in size from 1 to 1,000 microns across and from 20 to 1,000 microns deep, but preferably 80 to 500 microns across and 40 to 180 microns deep.

Next, in step 120, electrodes are applied or positioned (if not already in position) about the microporation(s) on the skin. This step involves the mechanical positioning of at least first and second electrodes such that at least one of the electrodes is electrically coupled to the micropore. That is, at least one of the electrodes (i.e., the first electrode) is positioned proximate the micropore so that the dominant or preferred current path to that electrode, induced by the electrical voltage from it and the second electrode is through the micropore. This assists in ensuring that at least some, if not the preferred, current density paths through the skin intersects at least some of the capillary loop structures and blood vessels present in these skins. The second electrode can be coupled to any other skin surface, acting to complete the current path through the skin with respect to the first electrode.

On the other hand, each of the first and second electrodes may be electrically coupled to micropores formed in the skin separated from each other. The electrodes may electrically penetrate into the micropore through a compliant electrolyte, e.g., a conductive hydrogel or a saline solution, placed on the contacting surface to facilitate electrical contact into the micropores. Additionally, according to a preferred embodiment, the same elements that are used to thermally microporate the skin are used as electroporation electrodes after the thermal microporation process has been completed.

Step 130, an optional step, involves deforming the skin surface so that it bows or bulges from the microporations. Depending on the depth of the micropores and the penetration of the electrode into them, a small deformation of the skin surface into a bowed shape is created from the micropores such that a line drawn from the micropores would intersect the targeted skin structures, such as capillaries and vessels in, for example, the dermis.

Next, in step 140, an electrical voltage pulse or series of voltage pulses is applied from the first and second electrodes of sufficient magnitude or amplitude such that the resulting current flow through the intervening tissue, including the targeted structures causes a potential drop across these targeted skin structures, such as capillary walls, which exceeds the electroporation threshold for these skin structures present in the current path. The pulsing scheme can include the modulation of pulse amplitude, pulse timing, pulse polarity and geometrical direction of the pulses to achieve desired electroporation effects. The duration of the pulse is relatively short, such as (1 μs to 10 ms) with an amplitude designed to ensure that the potential drop across the targeted membrane structures in the current path nominally exceeds a 1 volt potential, the value known in the art as being the nominal threshold level at which effective poration of a membrane begins to occur.

In step 150, biological fluid exuded from the microporated and electroporated skin is collected for analysis, or a substance, such as a drug or other bioactive agent is delivered into the permeability-enhanced skin.

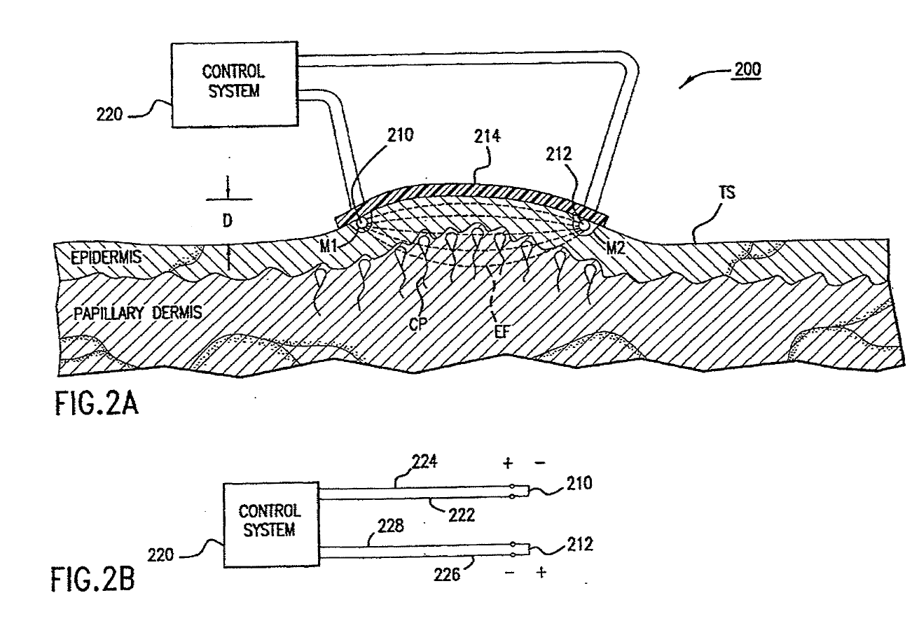

Turning to FIGS. 2A and 2B, an apparatus for electroporating and/or microporating and electroporating skin is described. Briefly, the apparatus comprises a heated probe suitable for conducting heat to a surface of the skin to form at least one micropore therein; at least first and second electrodes spaced apart from each other on the skin, with the first electrode being electrically coupled to the micropore; and control means for supplying energy to the heated element so as to form the at least one micropore, and for applying electrical voltage from the first and second electrodes for electroporating the skin.

Specifically, the apparatus, shown generally at reference numeral 200, comprises at least two electrodes 210 and 212. At least one of the electrodes is positioned in one of the micropores M1 and M2 formed through the skin surface TS, such as skin, and the other electrode is spaced from it and placed on the skin surface to complete the current path through the skin. Preferably, the electrodes 210 and 212 are placed in micropores M1 and M2. The electrodes 210 and 212 may be supported by a skin-contacting layer 214. Electrical voltage is applied from the electrodes 210 and 212 by energy supply means, included as part of a control system 220. The control system 220 includes the appropriate circuitry to supply electrical current and electrical voltage, and to control an optical energy source (if needed). Electrical contact of the electrodes 210 and 212 with the micropores can be achieved with a compliant electrolyte, such as a conductive hydrogel or a saline solution placed on the surface of the skin in the micropores. Once again, each electrode is preferably positioned proximate a micropore so as to be electrically coupled thereto.

The micropores M1 and M2 are formed prior to energization of the electroporation electrodes 210 and 212. These micropores may be formed in several ways, including thermal ablation via a heated probe by electrical or optical energy. Optical or laser thermal ablation involves placing a photosensitizing assembly, including an optically absorbing compound such as a dye, in contact with the surface of the skin, optical energy is focused on the photosensitizing assembly which heats it, and the heat is transferred to the surface of the skin, forming a micropore. In this case, a source of optical energy (not shown), controlled by the control system 220, is optically coupled to the photosensitizing assembly placed on the surface of the skin. Alternatively, the skin could be microporated using a laser which emits at a wavelength which is directly absorbed by the skin to be removed such as an excimer, holmium, erbium, or CO2 laser or the like. The use of direct laser absorption to form micropores is well known in the art. The application of the electroporation methods to which this invention is directed are suitably compatible with those other methods for forming the micropores in the skin.

In accordance with a preferred embodiment of the present invention, the electrodes 210 and 212 also serve as electrically heated probes used for thermally ablating the skin to form the micropores M1 and M2. Specifically, each electrode 210 and 212 comprises an electrically heated probe consisting of an electrically heated wire which is responsive to electrical current supplied therethrough. As shown in FIG. 2B, during the microporation stage or cycle, electrical current is coupled via conductor leads 222 and 224 to electrode 210 to supply an electrical current therethrough, and electrical current is also coupled via conductor lead lines 226 and 228 to electrode 212. On the other hand, during the electroporation stage or cycle, a voltage is applied to the conductors 222 and 224, relative to a voltage potential applied to conductors 226 and 228, causing electrode 210 to be at a positive potential with respect to electrode 212, or alternatively at a negative potential with respect to electrode 212 (depending on the polarity desired). Thus, the electrically heated probes described above are dual purpose in that they can perform the functions of microporation and of electroporation.

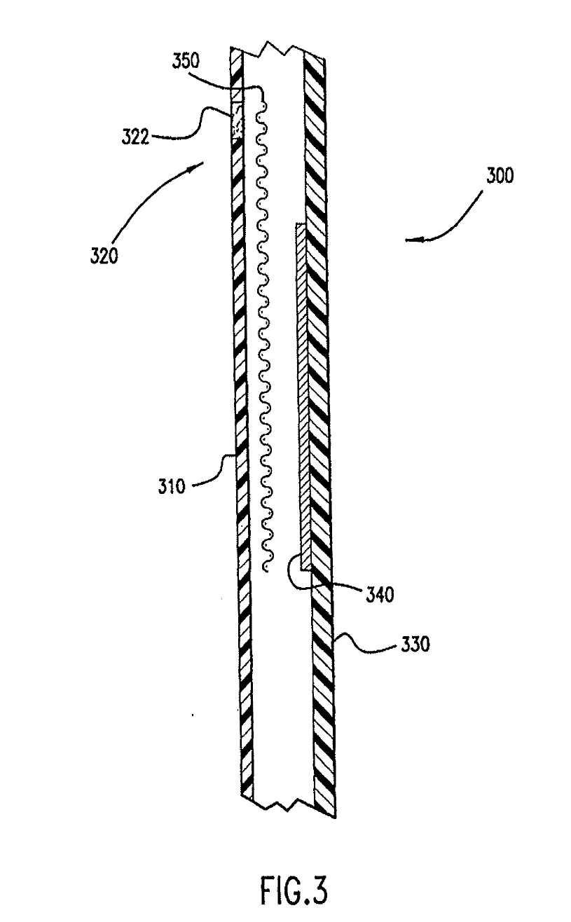

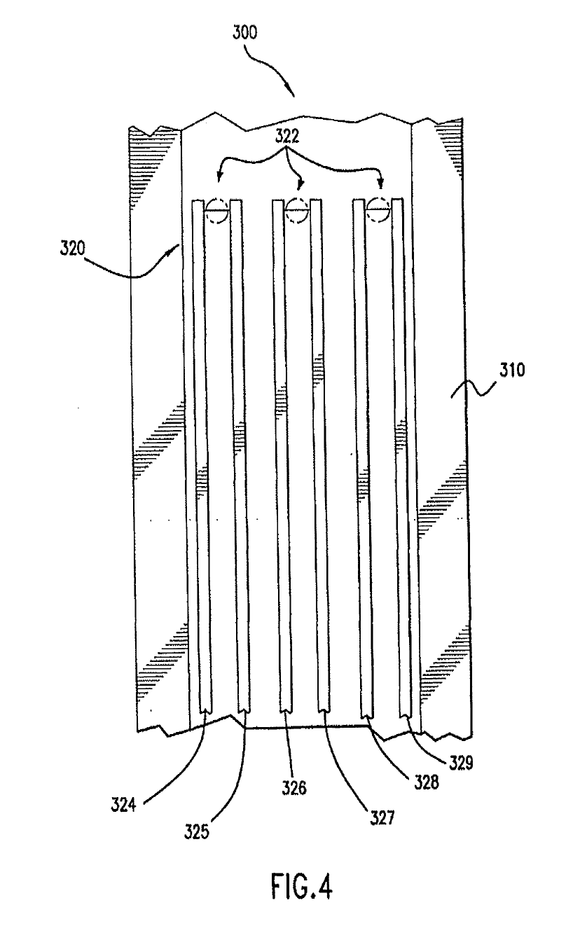

FIGS. 3 and 4 illustrate the incorporation of dual purpose electrically heated probes as part of an integrated fluid harvesting, collection and analysis device, shown generally at reference numeral 300. The device 300 includes a skin-contacting layer 310 having an electrically heated probe surface 320. The device 300 further comprises a detecting layer 340 such as a photometric sensor or an electrochemical biosensor, both of which are capable of providing an indication of a characteristic of a collected biological fluid, such as the level of an analyte in interstitial fluid. A meter (not shown) is coupled by a meter-interface layer 330, to the detecting layer 340 either electrically or optically, depending on the type of detecting layer used.

As shown in more detail in FIG. 4, the electrically heated probe surface 320 comprises several electrically heated probes 322 provided on the bottom surface of the skin-contacting layer 310. Three electrical heated probes 322 are shown, but any number of them may be provided. Each of the three heated probes 322 are connected to a pair of the electrical conductors 324, 325, 326, 327, 328 and 329 as shown. The electrical conductors extend the length of the skin-contacting layer 310 and terminate at a plurality of points near the lower end of the integrated device 300. Each electrically heated probe 322 is connected to a control system by the respective pairs of conductors {324, 325}, {326, 327} and {328, 329} as shown in FIG. 4.

Each electrically heated probe 322 can be activated individually through the appropriate selection and energization of the conductors 324, 325, 326, 327, 328 and 329. It may be advantageous to excite all electrically heated probes 322 simultaneously, thereby enabling either a series or parallel wiring design, reducing the number of interconnections to the device and facilitating a more rapid poration process. If only one electrically heated probe 322 is provided, then at least two conductors are provided for supplying electric current to it.

The electrically heated probes 322 function as solid thermal probes and are electrically heated so that a temperature of the skin, if skin, is raised to greater than 123 C. The electrically heated probes 322 comprise, for example a 100 to 500 micron long, 50 micron diameter, tungsten wire element. A number of human clinical studies have been performed wherein the surface microporation was achieved by using these types of wires as the electrically heated probe. These tungsten elements are typically laid flat against some form of a backing which naturally limits the depth of penetration of the wire element into the skin as it is being microporated (by virtue of the size of the element). The temperature of the heated element is modulated as needed to effect the microporation process.

A similar technique can be applied with the use of an optically heated probe in an integrated device, such as that disclosed in the co-pending application filed on even date. However, additional electrodes, such as those shown in the device of FIGS. 3 and 4, are additionally required in order to deliver the electroporation energy to the microporated skin. These additional electrodes can be conveniently formed on the lower surface of the photosensitizing assembly or layer using a lithographic process to create a printed circuit type pattern of conductive traces, portions of which serve as the electrodes on the skin-contacting side of this layer. This pattern of conductors registers the electrodes to the micropores to be formed, so that at least one electrode in the conductive trace is electrically coupled to a micropore.

Referring back to FIG. 2A, when a voltage is applied from the electrodes, so as to drive a current from them through the skin, current flux lines EF are created, such that at least one or more of them pass through the intervening targeted skin structures, such as the capillaries, CP in the skin. These flux lines (current paths) preferably go as deep as the papillary dermis so as to affect the capillaries therein. The voltage pulsing scheme may consist of a first voltage pulse of a polarity followed by a second voltage pulse of an opposite polarity. This causes current to flow in both directions from the electrodes. An advantage of redirecting the current flow in both directions at a given set of micropores is that by presenting the body with a balanced AC signal, no cumulative electrical polarization is established. This balanced signal has been shown to minimize the sensation to an individual.

Depending on the depth of the micropores and the penetration of the electrodes into them, the skin may be deformed by a predetermined amount D so as to further increase the number of electric flux lines that pass through specific targeted skin structures, such as capillaries in the skin. For example, the skin may be deformed by as much as 0.5 mm. This deformation could be achieved by several means. For example, the skin could be simply squeezed together from the microporations.

The use of electroporation coupled with microporation achieves significant advantages. Specifically, in the case of conventional electroporation, where pulses exceeding 50 to 150 volts are routinely used to electroporate the stratum corneum or mucosal layer, in the microporated skin environment of the present invention, pulses of only a few volts can be sufficient to electroporate the cell, capillary or other membranes within the targeted skin.

The manner in which the electroporation pulses are applied may vary. For example, a plurality of micropores spaced apart from each other in the skin may be formed, and the electrical pulses are then applied in multiple directions from different sets (pairs or more) of electrodes to facilitate the electroporation of a larger percentage of the area of the targeted structures in the intervening skin such as capillary walls. This multi-directional cross firing can be achieved with a plurality of electrically heated probes, similar to those shown in FIGS. 3 and 4. FIG. 8 illustrates such an embodiment, in which an 3.times.3 array 600 of electrically heated probes 610 is applied to the surface of the tissue. The electrically heated probes 610 also serve as the electroporation electrodes. The array can be formed using well known circuit printing technologies, such as etching, lithographic film deposition, etc. A suitable electrically heated poration element is then placed onto the appropriate conductors etched onto a circuit board/substrate. In this embodiment, all or selected ones of the heated probes 610 are energized to form micropores in the skin. Then, sets of the heated probes 610, which are already suitably electrically coupled to their respective micropores, are connected to a source of AC or DC voltage to create a current distribution from them. Voltage is applied from different sets of poration elements, now acting as electroporation electrodes, at the different micropores so as to change the direction of the electroporation through the skin. Successive pulses are preferably either in an opposite polarity with respect to the same set of electrodes and/or are from different sets of electrodes. Each possible path can be energized in either polarity, or toggled back and forth from polarities. The advantages of redirecting the current flow in both directions at a given set of micropores is that by presenting the body with a balanced AC signal, no cumulative electrical polarization is established. Furthermore, this multi-directional current control has been shown to dramatically reduce the sensation of the subject during the electroporation process as has the setting of the pulse parameters below certain peak voltage levels and with a duration of each pulse kept to a minimum, preferably under a few milliseconds.

It is well known in the art that electroporation can cause openings to form, temporarily, in the cell membranes and other internal skin membranes. By having breached the surface of the skin, such as the stratum corneum, mucosal layer or outer layer of a plant, and if desired the epidermis and dermis, or deeper into a plant, electroporation can be used with parameters tailored to act selectively on these underlying skin barriers. For any electromagnetic energy enhancement means, the specific action of the enhancement can be designed to focus on any part of the micropore, e.g., on the bottom of the micropore by focusing the discharge of the electrodes, phasing of multiple electrodes or other field forming methods and devices and the like. Alternatively, the enhancement can be focused more generally on the entire micropore or the area surrounding the pore.

The mode of operation of electroporation when applied after the microporation of the skin, has the advantage of being able to use operational parameters which would be useless for un-microporated, intact skin surface conditions. In particular, the operational settings useable when applied after the microporation of the skin or mucosal layer or the outer layer of a plant are generally close to those typically used in in vitro applications where single cell membranes are opened up for the delivery of a substance. Examples of these parameters are well known in the literature. For example, Sambvrook et al., Molecular Cloning: A Laboratory Manual, 2d Ed., Cold Spring Harbor Laboratory, Cold Spring Harbor, N.Y., 1989.

Still another enhancement which may be used in conjunction with the electroporation techniques described herein is the application of sonic energy.

The skin diseases which may be treated according to the invention include:

dermatological conditions associated with a keratinization disorder relating to differentiation and to proliferation, in particular acne, including common acne, comedo-type acne, polymorphic acne, rosacea, nodulocystic acne, acne conglobata, senile acne, and secondary acne such as solar, drug-related or occupational acne,

ichthyoses, ichthyosiform conditions, Darrier's disease, palmoplantar keratoderma, leukoplakia and leukoplakiform conditions, and cutaneous lichen,

dermatological conditions with an inflammatory immunoallergic component, with or without a cell proliferation disorder, in particular psoriasis, e.g. cutaneous, mucosal or ungual psoriasis, psoriatic rheumatism, cutaneous atopy, such as atopic dermatitis, eczema, respiratory atopy or gingival hypertrophy,

benign or malignant dermal or epidermal proliferations, of viral or non-viral origin, in particular common warts, flat warts, epidermodysplasia verruciformis, oral or florid papillomatoses, and T lymphoma,

proliferations which may be induced by ultraviolet light, in particular basal cell epithelioma and spinocellular epithelioma,

precancerous and cancerous skin lesions, in particular keratoacanthomas and melanoma,

immune dermatoses, in particular lupus erythematous,

bullous immune diseases,

dermatological symptoms of collagen diseases, such as scleroderma,

dermatological conditions with an immunological component,

skin disorders due to exposure to UV radiation, or light-induced or chronological aging of the skin, or actinic keratoses and pigmentations, in particular lentigines, or any pathologies associated with chronological or actinic aging, in particular xerosis,

sebaceous function disorders, in particular hyperseborrhoea acne or simple seborrhoea or seborrhoeic dermatitis,

cicatrization disorders or stretch marks,

pigmentation disorders, such as hyperpigmentation, melasma, chloasma, plane pigmented seborrheic warts, nevi, freckles, ephelides, actinic keratosis, hyperpigmentations with genetic determinism, hyperpigmentations of metabolic or medicamentous origin, melanoma, post-inflammatory hyperpigmentations in particular caused by abrasion, burn, scar, dermatitis, contact allergy, hyperpigmentations due to a skin trouble such as acne, psoriasis, rosacea, atopic dermatitis or all other hyperpigmented lesions, hypopigmentation or vitiligo,

alopecia of various origins, in particular alopecia caused by chemotherapy or radiation.

Acne, atopic dermatitis, psoriasis, rosacea, hyperpigmentation, melasma and melanoma are the preferred skin diseases which may be treated in the invention.

Among the substances which can be delivered in or though the skin according to the invention, mention may be made, by way of example, of agents for modulating the differentiation and/or proliferation and/or pigmentation of the skin, such as retinoic acid and isomers thereof, retinol and esters thereof, retinal, retinoids, in particular acitretin, etretinate, isotretinoin, and tretinoin, and compounds described in FR-2-570,377, EP-1 99,636, EP-325,540 and EP-402,072, rucinol, mequinol, retinol, vitamin D and derivatives thereof such as calcitriol, calcipotriol, corticosteroids such as fluocinolone acetonide, estrogens such as oestradiol, kojic acid or hydroquinone; anti-bacterial agents such as clindamycin phosphate, erythromycin or antibiotics of the tetracycline class; anti-parasitic agents, in particular metronidazole, ivermectin, crotamiton or pyrethrinoids; anti-fungal agents, in particular compounds belonging to the imidazole class, such as econazole, ketoconazole or miconazole, or salts and derivatives thereof; polyene compounds, such as amphotericin B; compounds of the allylamine family, such as terbinafine; compounds of the pyridinone family, such as cyclopirox; compounds of the morpholine family and derivatives, such as amorolfine; steroidal anti-inflammatories, such as hydrocortisone, anthralins (dioxyanthranol), anthranoids, betamethasone valerate or clobetasol 17-propionate, or non-steroidal anti-inflammatories, such as ibuprofen and salts or derivatives thereof, diclofenac and salts and derivatives thereof, acetylsalicylic acid, acetaminophen or glycyrrhetinic acid; anaesthetics such as lidocaine, lidocaine hydrochloride, tetracaine, pilocalne and derivatives thereof; anti-pruriginous agents such as thenaldine, trimeprazine or cyproheptadine; anti-viral agents such as acyclovir; keratolytic agents such as alpha- and beta-hydroxycarboxylic acids or beta-ketocarboxylic acids, salts, amides or esters thereof, and more particularly hydroxy acids such as glycolic acid, lactic acid, malic acid, salicylic acid, citric acid and fruit acids in general, and 5-n-octanoyl-salicylic acid; free-radical scavengers, such alpha-tocopherol or esters thereof, superoxide dismutases, certain metal-chelating agents or ascorbic acid and esters thereof; anti-seborrhoeic agents such as progesterone; anti-dandruff agents such as octopirox or zinc pyrithione; anti-acne agents such as retinoic acid, benzoyl peroxide or adapalene, tazarotene; anti-metabolites; agents for combating hair loss, such as minoxidil; antiseptics; as well as biologicals (e.g. hormones, peptides, antibodies or nucleic acids), the latter being particularly useful in treating psoriasis.

The active substance used in the invention may be employed alone or in combination.

Advantageously, the invention may employ compositions which comprise from 0.0001 to 20% by weight, relative to the total weight of the composition, of the active substance, preferably from 0.025 to 15% by weight, and more preferably from 0.01 to 5% by weight.

Of course, the amount of active agent in the compositions according to the invention will depend on the active agent under consideration.

The compositions will preferably comprise a substance from the group of clobetasol 17-propionate, adapalene, tazarotene, rucinol, retinoic acid, benzoyl peroxide, calcipotriol, calcitriol, ivermectin, terbinafine, amorolfine.

The pharmaceutical compositions may be in the form of ointments, creams, milks, salves, powders, impregnated pads, solutions, gels, sprays, lotions, suspensions, or in any convenient formulation for delivery through electroporation. They can also be in the form of microspheres or nanospheres or lipid vesicles or polymer vesicles or polymer patches and hydrogels allowing controlled release. Patches can be particularly advantageous to deliver the active substance.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for treating a skin disease, disorder or condition afflicting a patient in need thereof,
which method comprises
a) forming at least one micropore to a predetermined depth through a surface of the afflicted skin of said patient;
b) positioning at least a first electrode on the surface of the skin electrically coupled to the at least one micropore and a second electrode on the surface of the skin spaced apart from the first electrode;
c) applying an electrical voltage from the first and second electrodes to produce a desired electroporation in the skin,
and further comprising the step d) of delivering a biologically active species to the skin at the at least two micropores formed therein, wherein the said biologically active species is therapeutically/dermatologically active against said skin disease, disorder or condition.

2. The regime or regimen as defined by claim 1, wherein step c) comprises applying an electrical voltage of a sufficient magnitude from the first and second electrodes suitable to produce a potential drop exceeding a nominal threshold to achieve electroporation across the epithelial cell layer but not sufficient to electroporate membranes present in other skin structures, thereby achieving a selective electroporation of targeted membranes.

3. The regime or regimen as defined by claim 1, wherein step a) comprises forming first and second micropores spaced apart from each other, and wherein the first electrode is positioned to be electrically coupled to the first micropore, and the second electrode is positioned to be electrically coupled to the second micropore.

4. The regime or regimen as defined by claim 1, wherein step c) comprises applying a voltage pulse of a first polarity with respect to the first and second electrodes, followed by a voltage pulse of an opposite polarity with respect to the first and second electrodes.

5. The regime or regimen as defined by claim 1, wherein step a) comprises forming a plurality of micropores spaced apart from each other in the skin, and step b) comprises placing a plurality of electrodes each being electrically coupled to a different one of the micropores, and wherein step c) comprises applying electrical voltage pulses from different sets of the plurality of electrodes so as to electroporate the skin in multiple directions.

6. The regime or regimen as defined by claim 5, wherein step c) comprises applying a voltage pulse of a first polarity from a first set of electrodes followed by a voltage pulse of an opposite polarity from the first set of electrodes.

7. The regime or regimen as defined by claim 1, wherein step a) comprises applying a photosensitizing material to the surface of the afflicted skin and irradiating the photosensitizing material with optical energy, whereby the photosensitizing material is responsive to the optical energy so as to heat up and conductively transfer heat to the surface of the skin to form the at least one micropore.

8. The regime or regimen as defined by claim 1, further comprising the step e) of deforming the surface of the afflicted skin from the first and second electrodes such that the surface of the afflicted skin sufficiently bulges from the first and second electrodes to place skin structures desired to be electroporated in a principal current path from the first and second electrodes.

9. The regime or regimen as defined by claim 1, wherein step a) comprises applying a photosensitizing material to the surface of the afflicted skin and irradiating the photosensitizing material with optical energy, whereby the photosensitizing material is responsive to the optical energy such as to heat up and conductively transfer heat to the surface of the afflicted skin to form the at least one micropore, whereby the step of positioning the first and second electrodes comprises positioning conductive traces on a skin-contacting side of the photosensitizing material registered with the micropores.

10. The regime or regimen as defined by claim 1, wherein said disease, disorder or condition is selected from the group consisting of:
dermatological conditions associated with a keratinization disorder relating to differentiation and to proliferation, acne, common acne, comedo-type acne, polymorphic acne, rosacea, nodulocystic acne, acne conglobata, senile acne, and secondary acne, solar, drug-related or occupational acne;
ichthyoses, ichthyosiform conditions, Darrier's disease, palmoplantar keratoderma, leukoplakia and leukoplakiform conditions, cutaneous lichen;
dermatological conditions with an inflammatory immunoallergic component, with or without a cell proliferation disorder, psoriasis, cutaneous, mucosal or ungual psoriasis, psoriatic rheumatism, cutaneous atopy, atopic dermatitis, eczema, respiratory atopy or gingival hypertrophy,
benign or malignant dermal or epidermal proliferations, of viral or non-viral origin, common warts, flat warts, epidermodysplasia verruciformis, oral or florid papillomatoses, T lymphoma,
proliferations which may be induced by ultraviolet light, basal cell epithelioma and spinocellular epithelioma,
precancerous and cancerous skin lesions, keratoacanthomas and melanoma,
immune dermatoses, lupus erythematous,
bullous immune diseases,
dermatological symptoms of collagen diseases, scleroderma,
dermatological conditions with an immunological component,
skin disorders due to exposure to UV radiation, or light-induced or chronological aging of the skin, or actinic keratoses and pigmentations, lentigines, or any pathologies associated with chronological or actinic aging, xerosis,
sebaceous function disorders, hyperseborrhoea acne or simple seborrhoea or seborrhoeic dermatitis,
cicatrization disorders or stretch marks,
pigmentation disorders, hyperpigmentation, melasma, chloasma, plane pigmented seborrheic warts, nevi, freckles, ephelides, actinic keratosis, hyperpigmentations with genetic determinism, hyperpigmentations of metabolic or medicamentous origin, melanoma, post-inflammatory hyperpigmentations, those caused by abrasion, burn, scar, dermatitis, contact allergy, hyperpigmentations due to a skin trouble, psoriasis, rosacea, atopic dermatitis all other hyperpigmented lesions, hypopigmentation or vitiligo, and
alopecia of various origins, and alopecia caused by chemotherapy or radiation.

11. The regime or regimen as defined by claim 10, wherein said disease, disorder or condition is selected from the group consisting of acne, atopic dermatitis, psoriasis, rosacea, hyperpigmentation, melasma and melanoma.

12. The regime or regimen as defined by claim 11, wherein said disease, disorder or condition comprises acne.

13. The regime or regimen as defined by claim 11, wherein said disease, disorder or condition comprises psoriasis.

14. The regime or regimen as defined by claim 11, wherein said disease, disorder or condition comprises melasma.

15. The regime or regimen as defined by claim 11, wherein said disease, disorder or condition comprises rosacea.

16. The regime or regimen as defined by claim 1, wherein said disease, disorder or condition comprises melanoma.

17. The regime or regimen as defined by claim 1, wherein said biologically active species is selected from the group consisting of a retinoid, vitamin D and derivative thereof, a corticosteroid, an estrogen, an antibacterial agent, an anti-parasitic agent, an anti-fungal agent, a polyene compound, compounds of the allylamine family, compounds of the pyridinone family, steroidal anti-inflammatories, non-steroidal anti-inflammatories, anaesthetics; antiseptics; anti-pruriginous agents, anti-viral agents; keratolytic agents; free-radical scavengers, anti-seborrhoeic agents; anti-dandruff agents; anti-acne agents, anti-metabolites; agents for combating hair loss; and biologicals.

18. The regime or regimen as defined by claim 1, wherein said biologically active species is selected form the group consisting of estradiol, calcitriol, calcipotriol, fluocinolone acetonide, kojic acid, hydroquinone, clindamycin phosphate, erythromycin, antibiotics of the tetracycline class, metronidazole, ivermectin, crotamiton, pyrethrinoids, econazole, ketoconazole, miconazole, or salts and derivatives thereof, amphotericin B, terbinafine, cyclopirox, amorolfine, hydrocortisone, dioxyanthranol, anthranoids, betamethasone valerate, clobetasol 17-propionate, ibuprofen and salts or derivatives thereof, diclofenac and salts and derivatives thereof, acetylsalicylic acid, acetaminophen, glycyrrhetinic acid, lidocaine, lidocaine hydrochloride, tetracaine, pilocalne and derivatives thereof; thenaldine, trimeprazine or cyproheptadine, acyclovir, glycolic acid, lactic acid, malic acid, salicylic acid, citric acid and fruit acids, 5-n-octanoyl-salicylic acid, alpha-tocopherol or esters thereof, superoxide dismutases, ascorbic acid and esters thereof, progesterone, octopirox, zinc pyrithione; retinoic acid, benzoyl peroxide, adapalene, acitretin, etretinate, isotretinoin, tretinoin, tazarotene, compounds described in FR-2-570,377, EP-1 99,636, EP-325,540 and EP-402,072, rucinol, mequinol, retinol, minoxidil, hormones, peptides, antibodies and nucleic acids.

19. A regime or regimen for treating a skin disease, disorder or condition afflicting a patient in need thereof, which method comprises:
a) forming at least one micropore to a predetermined depth through a surface of the skin by placing an electrically heated probe at the surface of the afflicted skin and supplying electrical current to the electrically heated probe so as to ablate the surface of the afflicted skin to form the at least one micropore;
b) positioning at least a first electrode electrically coupled to the at least one micropore and a second electrode spaced apart from the first electrode;
c) applying an electrical voltage from the first and second electrodes to produce a desired electroporation in the skin,
and further comprising the step d) of delivering a biologically active species to the skin at the at least two micropores formed therein, wherein said biologically active species is therapeutically/dermatologically active against said disease, disorder or condition.

20. The regime or regimen as defined by claim 19, wherein the electrically heated probe also serves as the first electrode such that the electrical voltage is applied from the electrically heated probe and the second electrode.

21. A regime or regimen for treating a skin disease, disorder or condition afflicting a patient in need thereof, which method comprises:
a) forming at least one micropore to a predetermined depth through a surface of the afflicted skin by placing first and second electrically heated probes at the surface of the skin spaced apart from each other and supplying electrical current to each of the first and second electrically heated probes such as to ablate the surface of the afflicted skin in order to form two micropores spaced apart from each other;
b) positioning at least a first electrode electrically coupled to the at least one micropore and a second electrode spaced apart from the first electrode;
c) applying an electrical voltage from the first and second electrodes to produce a desired electroporation in the afflicted skin.
and further comprising the step d) of delivering a biologically active species to the skin at the at least two micropores formed therein, wherein the biologically active species is therapeutically/dermatologically active against said disease, disorder or condition.
